(19) United States
(12) Patent Application Publication
(10) Pub. No.: US 2006/0111686 A1
(43) Pub. Date: May 25, 2006

(54) Absorbent article with heat deactivated area

(76) Inventor: Uwe Schneider, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/995,863

(22) Filed: Nov. 23, 2004

(51) Int. Cl. A61F 13/15

(52) U.S. Cl. 604/385.26; 604/382

(57) ABSTRACT

A disposable absorbent article has a chassis which has a first longitudinal edge, a second longitudinal edge, a front waist edge, a rear waist edge, a front waist region, a rear waist region, and a crotch region disposed therebetween. A first elastic member extending proximate to the first longitudinal edge from about the front waist edge to about the rear waist edge is joined under tension to the chassis such that corrugations are formed in the chassis inboard of the first elastic member in the front, rear, and crotch regions. A first heat deactivated zone overlaps the first elastic member in at least a portion of the front waist region such that corrugations are minimized in the front waist region adjacent to the front waist edge when the first elastic member is in a relaxed state.

### FIELD OF THE INVENTION

The present invention relates to disposable absorbent articles such as diapers, incontinent briefs, training pants, and the like, and more particularly, to absorbent articles that experience heat deactivation in at least a portion of the absorbent article.

### BACKGROUND OF THE INVENTION

Infants and other incontinent individuals wear absorbent articles such as diapers to receive and contain urine and other body exudates. A typical absorbent article can have a front waist region, a rear waist region, and a crotch region disposed between the front waist region and the rear waist region. In order to minimize leakage in the absorbent article, elastics may be utilized in many regions of the absorbent article.

For example, in an effort to control leakage from absorbent articles about the legs of a wearer, leg elastics may be incorporated into the absorbent article. The leg elastics can extend from the front waist region to the rear waist region of the article during processing. Also during processing, the leg elastics are typically prestrained prior to being joined to the article in order to provide a better fit about the legs of the wearer. Because the leg elastics are prestrained, corrugations can be created in the article upon relaxation of the leg elastics.

In another example, elastic waist features can be incorporated into the front waist region and the rear waist region of the article in order to more snugly fit the article about the wearer's waist. The elastic waist feature is typically an elastomeric laminate which has an elastic member bonded to a substrate. Typically, the elastic waist feature is prestrained prior to being joined to the absorbent article. Because the elastic waist feature is prestrained, corrugations can be created in the front waist region or the rear waist region of the article upon relaxation of the elastic waist feature.

The corrugations caused by either the leg elastics or the elastic waist feature can allow for a more snug fitting absorbent article. However, these corrugations are not necessarily desired in every portion of the absorbent article in which they occur. For instance, while corrugations may be desired in the crotch region of the absorbent article, corrugations may be undesirable in the front and rear waist regions. For example, corrugations created by the leg elastics in the front waist region or the rear waist region may tend to cause the absorbent article to fit poorly about the waist of the wearer. Also, the leg elastics and elastic waist features can create corrugations in either the front waist region or the rear waist region that generally are not aesthetically pleasing to a caregiver.

A method for eliminating the corrugations caused by the leg elastics in the front and rear waist regions is to strategically bond the leg elastics to the absorbent article. For example, if corrugations were desired in the crotch region but not the front waist region or the rear waist region, the leg elastics could be adhesively bonded in the crotch region and substantially unbonded in the front and rear waist regions. Thus, upon relaxation of the leg elastics, ends of the leg elastics in the front waist region would recede into the absorbent article toward the crotch region, and ends of the leg elastics near the rear waist region would also recede into the absorbent article toward the crotch region. Because the leg elastics are substantially unbonded in the front waist region and the rear waist region, upon receding into the absorbent article, the leg elastics would not cause the absorbent article to contract in the front waist region or the rear waist region thereby minimizing corrugations in these regions.

Unfortunately, when the leg elastics are substantially unbonded in the front and rear waist regions, weak areas may occur in the article. For example, when an ear panel is formed from a topsheet, a backsheet, a barrier leg cuff, or combinations thereof, a portion of the topsheet, backsheet, or barrier leg cuff, remains unbonded to either the leg elastics or any other element in the article in these unbonded regions because of the absence of adhesive. These unbonded regions devoid of adhesive are typically referred to as “glue open channels” and can cause the ear panels of the absorbent article to be weakened.

Elastic waist features can cause corrugations in the front waist region and the rear waist region which are generally not aesthetically pleasing. For instance, if a graphic design is desired on the front waist region, the graphic design can be placed on the article when the front waist region is substantially free of corrugations. However, if subsequently the elastic waist feature is joined to the front waist region under tension, substantial corrugations could result in both the graphic design and the article in the front waist region once the elastic waist feature is relaxed. Alternatively, if the elastic waist feature was joined to the front waist region under tension prior to the graphic design being added to the article, upon relaxation of the elastic waist feature, corrugations could form in the front waist region. These corrugations could make adding the graphic design to the absorbent article difficult.

Consequently, a need exists for an absorbent article which has minimal undesired corrugations while maintaining corrugations that are desirable. Also, a need exists for an absorbent article which has a reinforced area where undesired corrugations are minimized.

### SUMMARY OF THE INVENTION

The present invention minimizes undesired corrugations in a disposable absorbent article while maintaining corrugations that are desirable. The present invention can also provide an aesthetically pleasing absorbent article and can provide an absorbent article with reinforced ear panels. In addition, the present invention can also provide an absorbent article which has a waist feature with varying characteristics from a front waist region to a back waist region.

In one embodiment, a disposable absorbent article can comprise a chassis having a first longitudinal edge, a second longitudinal edge, a front waist edge, a rear waist edge, a front waist region, a rear waist region, and a crotch region disposed therebetween. The chassis may further comprise a topsheet, a backsheet joined to the topsheet, and an absorbent core disposed between the topsheet and the backsheet.

The disposable absorbent article may further comprise a first elastic member joined to the chassis proximate the first longitudinal edge under tension such that corrugations are formed in the chassis in the front, rear, and crotch regions. The corrugations are inboard of the first elastic member upon relaxation of the first elastic member. A first heat deactivated zone can overlap the first elastic member in at least a portion of the front waist region or the rear waist region thereby minimizing the corrugations in the front or rear waist region.

In another embodiment, a disposable absorbent article can comprise the chassis described above but wherein the front, rear, and crotch regions each include a first lateral region, a second lateral region, and a middle region between the first and second lateral regions. The disposable absorbent article may further comprise an elastomeric first waist member joined under tension to the front waist region of the chassis such that upon relaxation corrugations form in the front waist region. The elastomeric first waist member comprises a first waist heat deactivated zone overlapping the elastomeric first waist member in the middle region of the front waist region such that upon relaxation, corrugations in the middle region of the front waist region are minimized.

A method for making a disposable absorbent article, can comprise the step of providing a chassis similar to that described above. A first elastic member and a second elastic member are joined to the chassis under tension proximate to the first longitudinal edge and second longitudinal edge, respectively, such that corrugations are formed in the front, rear, and crotch regions inboard of the first elastic member and second elastic member upon relaxation of the elastic members. The method further comprises the steps of heat deactivating a portion of the first elastic member and a portion of the second elastic member while under tension proximate to the front waist edge such that corrugations near the front waist edge are minimized.

In another embodiment, a method for making a disposable absorbent article comprises the step of providing a chassis similar to that described above. A first waist member comprising an elastic member can be joined under tension to a substrate such that upon relaxation, the first waist member corrugates. A portion of the first waist member over the first elastic member can be heat deactivated while under tension such that corrugations in the portion of the first waist member are minimized when the first waist member is relaxed. Also, the first waist member can be joined to the front waist region under tension such that in a relaxed state corrugations in the front waist region are minimized proximate the portion of the first waist member.

### BRIEF DESCRIPTION OF THE DRAWINGS

FIG. 1A is a cut-away plan view of an absorbent article that can be modified in accordance with the claimed invention.

FIG. 1B is a plan view of the absorbent article of FIG. 1A showing resulting corrugations caused by a first and a second elastic member.

FIG. 1C is a plan view of the absorbent article of FIG. 1A showing minimized corrugations in a front waist region.

FIG. 1D is an embodiment of the absorbent article of FIG. 1A as seen in a cross sectional view of through line 1D-1D.

FIG. 1E is another embodiment of the absorbent article of FIG. 1A as seen in a cross sectional view through line 1D-1D.

FIG. 2A is a plan view of an absorbent article in a flattened condition that can be modified in accordance with the claimed invention.

FIG. 2B is a plan view of the absorbent article of FIG. 2A showing an embodiment of the claimed invention.

FIG. 2C is a schematic view of the absorbent article of FIG. 2A assembled into a pant-type article.

FIG. 3A is a schematic view of a plurality of absorbent articles on a web which has discrete elastomeric members joined thereto.

FIG. 3B is a plan view of an absorbent article of FIG. 3A assembled in accordance with the claimed invention.

FIG. 4 is a plan view of a discrete elastomeric laminate shown in FIG. 3A.

FIG. 5 is a plan view of an absorbent article showing a diaper chassis having a plurality of regions.

FIG. 6A is a perspective view of a pull-on diaper in accordance with the invention.

FIG. 6B is a plan view of the pull-on diaper of FIG. 6A in a flattened condition.

### DETAILED DESCRIPTION OF THE INVENTION

Definitions:

As used herein, the following terms have the following meanings:

“Absorbent article” refers to devices that absorb and contain liquid, and more specifically, refers to devices that are placed against or in proximity to the body of the wearer to absorb and contain the various exudates discharged from the body.

The terms “corrugations” or “ruggosities” are used to describe hills and valleys that occur in a substrate or in a laminate structure. The hills or valleys that occur are not necessarily uniform in nature. The corrugations or ruggosities extend inboard of an elastic member into the chassis of the absorbent article. Depending on the orientation of the elastic member, the corrugations or ruggosities can extend inboard into the chassis in either a longitudinal or lateral direction.

As used herein, the term “diaper” refers to an absorbent article generally worn by infants and incontinent persons about the lower torso.

The term “disposable” is used herein to describe absorbent articles that generally are not intended to be laundered or otherwise restored or reused as an absorbent article (i.e., they are intended to be discarded after a single use and, preferably, to be recycled, composted or otherwise disposed of in an environmentally compatible manner).

As used herein, the term “disposed” is used to mean that an element(s) is formed (joined and positioned) in a particular place or position as a unitary structure with other elements or as a separate element joined to another element.

An “elastic,” “elastomer” or “elastomeric” refers to polymers or laminates exhibiting elastic properties. They include any material that upon application of a force to its relaxed, initial length, can stretch or elongate to an elongated length of more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Longitudinal” is a direction running parallel to the maximum linear dimension of the article and includes directions within ±45° of the longitudinal direction.

The “lateral” or “transverse” direction is orthogonal to the longitudinal direction.

The term “pant”, as used herein, refers to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the term “pant” is used herein, pants are also commonly referred to as “closed diapers”, “prefastened diapers”, “pull-on diapers”, “training pants” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999.

Description:

An absorbent article with a heat deactivated zone is described herein. The heat deactivated zone can be located on the absorbent article in any region of the article thereby minimizing undesired corrugations in the absorbent article while maintaining the corrugations that are desired. By minimizing the undesired corrugations, the heat deactivated zone can provide an aesthetically pleasing absorbent article. In addition, the heat deactivated zone can minimize the undesired corrugations without causing weakened areas in the absorbent article.

Heat generally can break or relax an elastic member thereby deactivating the elastic member. A heat deactivated zone is created where the heat was applied to the elastic member. An elastic member has a tensile strength which can vary with the chemistry of the member, the thickness of the member, etc. When an elastic member is extended via a tension force, the elastic member exerts a contraction force counteracting the tension force. The elastic member is extended until the contraction force equals the tension force. If the tension force exceeds the tensile strength of the elastic member, then the elastic member breaks. Without wishing to be bound by theory, it is believed that while under tension, if heat is applied to the elastic member, the tensile strength can decrease and become less than the contractive force of the elastic member in the area of applied heat. When this occurs, the elastic member in the area of the applied heat is broken.

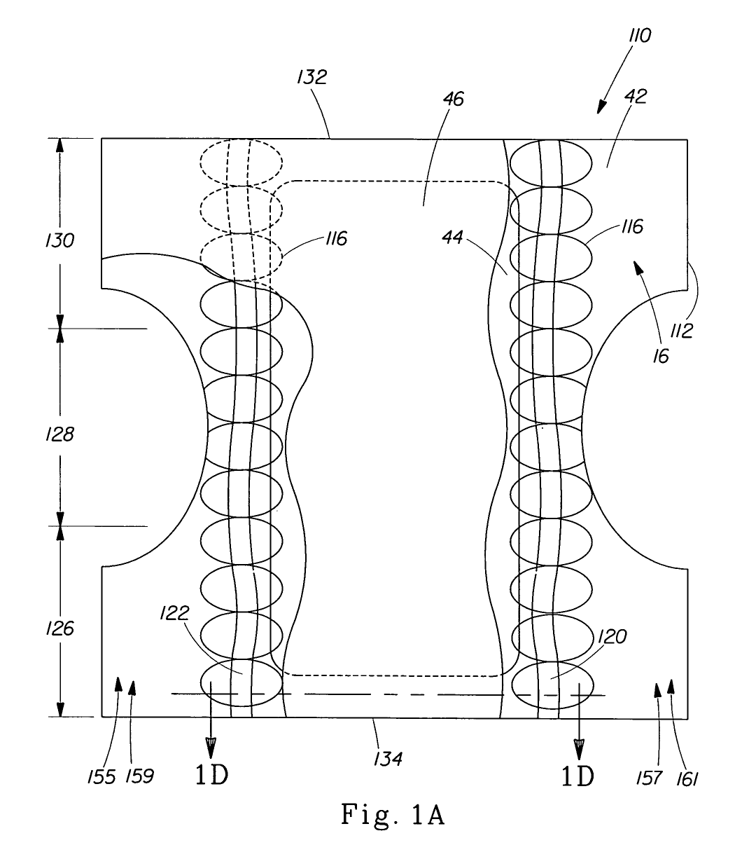

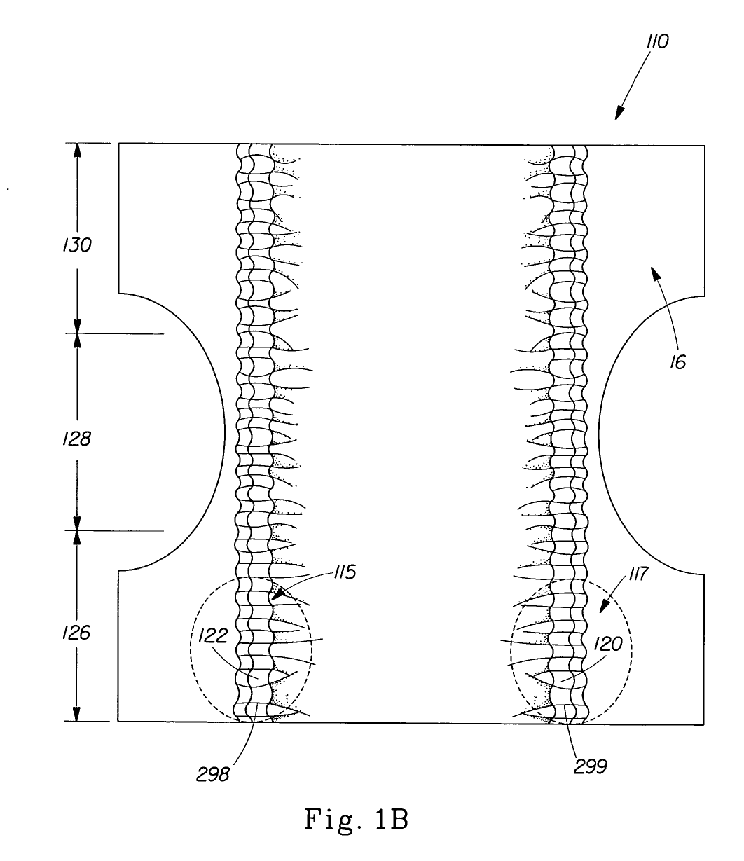

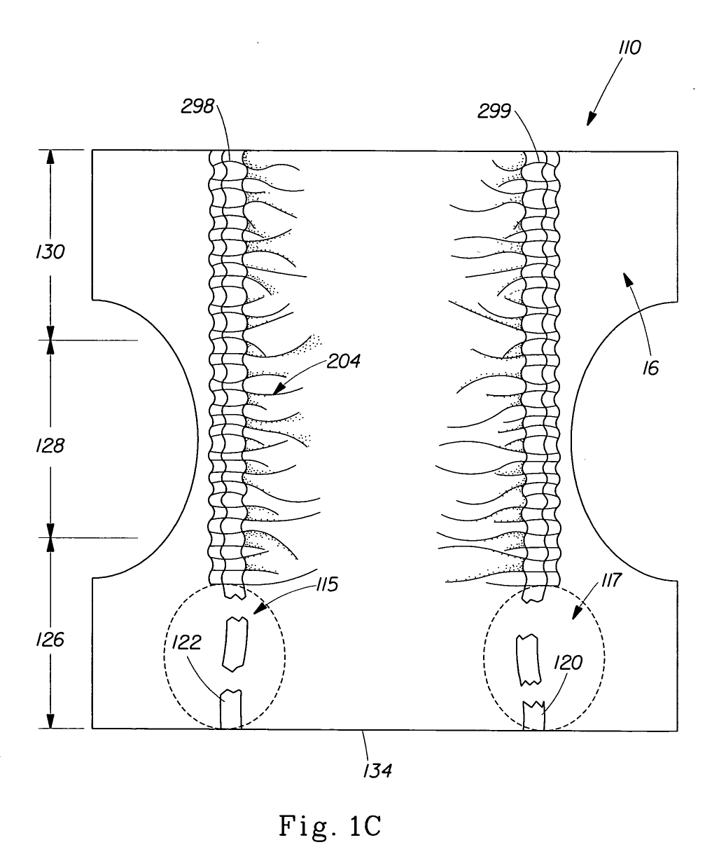

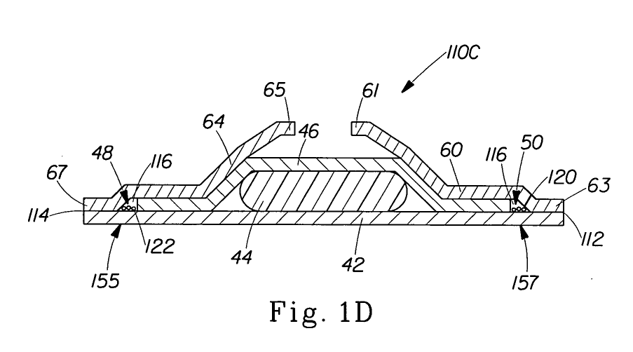

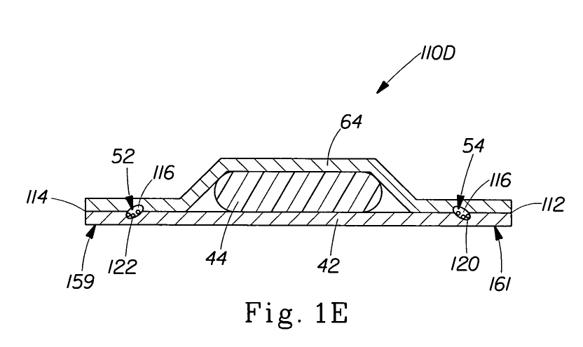

As shown in FIG. 1A, an absorbent article 110 has a chassis 16 which comprises a front waist region 126 which has a front waist edge 134, and a rear waist region 130 which has a rear waist edge 132. A crotch region 128 is disposed between the front waist region 126 and the rear waist region 130. The chassis 16 further comprises a topsheet 46, a backsheet 42 joined to the topsheet 46 and an absorbent core 44 disposed between the topsheet 46 and the backsheet 42.

The absorbent article 110 further comprises a first elastic member 122 which extends from about the front waist edge 134 to about the rear waist edge 132 proximate to a first longitudinal edge 114 of the chassis 16. Similarly, a second elastic member 120 can extend from about the front waist edge 134 to about the rear waist edge 132 proximate to a second longitudinal edge 112 of the chassis 16.

Both the first elastic member 122 and the second elastic member 120 can be prestrained and then joined to the chassis 16 by adhesive 116. The adhesive 116 may join the first elastic member 122 to the chassis 16 in the front waist region 126 proximate to the front waist edge 134. Similarly, the adhesive 116 may join the first elastic member 122 to the chassis 16 in the rear waist region 130 proximate to the rear waist edge 132. The second elastic member 120 may also be joined to the chassis 16 by the adhesive 116 proximate to the front waist edge 134 and the rear waist edge 132. The adhesive 116 can be continuous throughout the front waist region 126 and the rear waist region 130 for both the first elastic member 122 and the second elastic member 120.

As shown in FIG. 1B, if the first elastic member 122 and the second elastic member 120 are bonded substantially in the front waist region 126, the rear waist region 130, and the crotch region 128 while under tension, i.e. prestrained, corrugations 298, 299 can occur in the front waist region 126, the rear waist region 130, and the crotch region 128, upon relaxation of the first elastic member 122 and the second elastic member 120. The corrugations 298, 299 are generally formed inboard of the first and second elastic members 122, 120. Also, as mentioned previously, corrugations 298, 299 in either the front waist region 126 or the rear waist region 130 are generally undesirable. Thus, in order to minimize corrugations in the front waist region 126, the absorbent article 110 can comprise a first heat deactivated zone 115 and a second heat deactivated zone 117.

The first heat deactivated zone 115 can overlap and relax the first elastic member 122 in a portion of the front waist region 126 such that the corrugations in the front waist region 126 are minimized. Similarly, the second heat deactivated zone 117 can overlap and relax the second elastic member 120 in a portion of the front waist region 126 such that corrugations are minimized. In a particular embodiment, the absorbent article 110 may further comprise a third and a fourth heat deactivated zone which minimize corrugations in the rear waist region 130 created by the first and second elastic members 122, 120. By minimizing the corrugations caused by the first elastic member 122 and the second elastic member 120 in the front waist region 126 and in the rear waist region 130, a more aesthetically pleasing absorbent article can be provided.

FIG. 1C shows the effect of the first and second heat deactivated zones 115, 117 on the absorbent article 110. Both the first elastic member 122 and the second elastic member 120 are joined to the absorbent article proximate to the front waist edge 134. Note that the front waist region 126 is substantially free of corrugations while the crotch region 128 comprises transverse corrugations 204 which extend inboard of the first and second elastic members 122, 120.

Alternatively, the absorbent article 110 could comprise heat deactivated zones which are applied to the crotch region 128; however, corrugations 298, 299 in the crotch region 128 may be desirable. As an example, if the first and second elastic members 122, 120 have a tension force of between 0.29 Newtons and 0.69 Newtons applied to each, corrugations in the crotch region 128 transversely inboard of the first elastic member 122 and transversely inboard of the second elastic member 120 may occur. These transverse corrugations 204 in the crotch region 128, inboard of the first and second elastic members 122, 120, help distribute exudates from a wearer to different areas of an absorbent core. By distributing exudates to different areas of the absorbent core, the exudates are absorbed into the absorbent core more rapidly than they would if merely concentrated in a single location on the absorbent core.

The amount of tension force required to create transverse corrugations 204 in the crotch region 128 depends on many factors. For instance, an absorbent article with a thicker absorbent core may require more tension force in the first and second elastic members 122, 120 than would an absorbent article with a thinner absorbent core in order to accomplish the transverse corrugations 204 in the crotch region 128. In addition, if the transverse corrugations 204 are desired to extend from the first elastic member 122 to the second elastic member 120, then a higher tension force may be required. In this embodiment, sufficient tension should be applied to the first and second elastic members 122, 120, such that transverse corrugations inboard of the elastic members are created in the crotch region 128.

In addition to minimizing corrugations in the front and rear waist regions 126, 130, thereby providing a more aesthetically pleasing absorbent article, the present invention, in accordance with the absorbent article of FIG. 1A, can also provide the absorbent article with a reinforced ear panel. Embodiments of absorbent articles with reinforced ear panels are discussed below as examples.

As shown in FIG. 1D, an absorbent article 110C can comprise barrier leg cuffs 60 and 64. The barrier leg cuffs 60 and 64 comprise distal ends 61, 65 and proximal ends 63, 67, respectively. A topsheet 46 can also be joined to the backsheet 42 interior of each barrier leg cuff 60 and 64, and an absorbent core 44 resides between the topsheet 46 and the backsheet 42.

Barrier leg cuff 64 can extend laterally outward from the absorbent core 44, and a portion of the barrier leg cuff 64 near the proximal end 67 can be joined to the backsheet 42 such that a first ear panel 155 is formed (see also FIG. 1A). The first elastic member 122 can extend through a first opening 48 in the first ear panel 155 which can be formed by joining of the barrier leg cuff 64 to the backsheet 42 and by joining the topsheet 46 to the backsheet 42. As discussed previously, upon relaxation of the first elastic member 122, corrugations can form in the front waist region; however, because the absorbent article comprises a first heat deactivated zone overlapping the first elastic member 122, the first elastic member 122 does not need to recede into the absorbent article 110C in order to minimize the corrugations in the front waist region. Instead, the first heat deactivated zone minimizes the corrugations created by the first elastic member 122 in the front waist region. Therefore, the absorbent article 110C can comprise adhesive 116 in the first opening 48 in the front waist region proximate to the front waist edge which precludes a glue open channel. The adhesive 116 can join the barrier leg cuff 64, the backsheet 42, and the first elastic member 122, in the front waist region thereby reinforcing the first ear panel 155.

Similarly, barrier leg cuff 60 can extend laterally outward from the absorbent core 44 and a portion of the barrier leg cuff 60 near the proximal end 63 can be joined to the backsheet 42 such that a second ear panel 157 is formed (see also FIG. 1A). The second elastic member 120 can extend through a second opening 50 in the second ear panel 157. The second opening 50 can be formed in a similar manner as the first opening 48 discussed above. Similar to the first heat deactivated zone, the second heat deactivated zone minimizes the corrugations created by the second elastic member 120 in the front waist region thereby reinforcing the second ear panel 157.

As shown in FIG. 1E, an absorbent article 110D can comprise a topsheet 64 and the backsheet 42. The absorbent core 44 can be disposed in between the topsheet 64 and the backsheet 42. The topsheet 64 can extend laterally outward from the absorbent core 44 and can be joined to the backsheet 42 such that a third ear panel 159 is formed (see also FIG. 1A). The first elastic member 122 can extend through a third opening 52 in the third ear panel 159 which can be formed by joining the topsheet 64 to the backsheet 42. As discussed previously, upon relaxation of the first elastic member 122, corrugations can form in the front waist region; however, because the absorbent article comprises a first heat deactivated zone overlapping the first elastic member 122, the first elastic member 122 does not need to recede into the absorbent article 110D in order to minimize the corrugations in the front waist region. Instead, the first heat deactivated zone minimizes the corrugations caused by the first elastic member 122 in the front waist region. Therefore, the absorbent article 110D can comprise adhesive 116 in the third opening 52 in the front waist region proximate to the front waist edge which precludes a glue open channel. The adhesive 116 can join the topsheet 64 to the backsheet 42 and the first elastic member 122 in the front waist region thereby reinforcing the third ear panel 159.

Similarly, the topsheet 64 can extend laterally outward from the absorbent core 44 and can be joined to the backsheet 42 such that a fourth ear panel 161 is formed (see also FIG. 1A). The second elastic member 120 can extend through a fourth opening 54 in the fourth ear panel 161 which can be formed by joining the topsheet 64 to the backsheet 42. Similar to the first heat deactivated zone, the second heat deactivated zone minimizes the corrugations caused by the second elastic member 120 in the front waist region thereby reinforcing the fourth ear panel 161.

In addition, to the first and second heat deactivated zones discussed above, a third and a fourth heat deactivated zone can minimize the corrugations created by the first and second elastic members 122, 120 in the rear waist region. Thus, if ear panels extending outboard of the absorbent core 44 exist in the rear waist region, then the third and fourth heat deactivated zones can eliminate the corrugations in the rear waist region and reinforce the ear panels in the rear waist region.

As mentioned previously, corrugations can also be caused by elastic waist features in addition to leg elastics. Thus, alternative to or in conjunction with the heat deactivated zones pertaining to leg elastics, the present invention contemplates a waist heat deactivated zone which deactivates a portion or all of an elastic waist feature. The embodiments discussed below merely provide examples of absorbent articles with the waist heat deactivated zone which affects an elastic waist feature. The waist heat deactivated zones may be positioned at any location on the waist members discussed below, or at any location on the article where a desire to minimize corrugations exists.

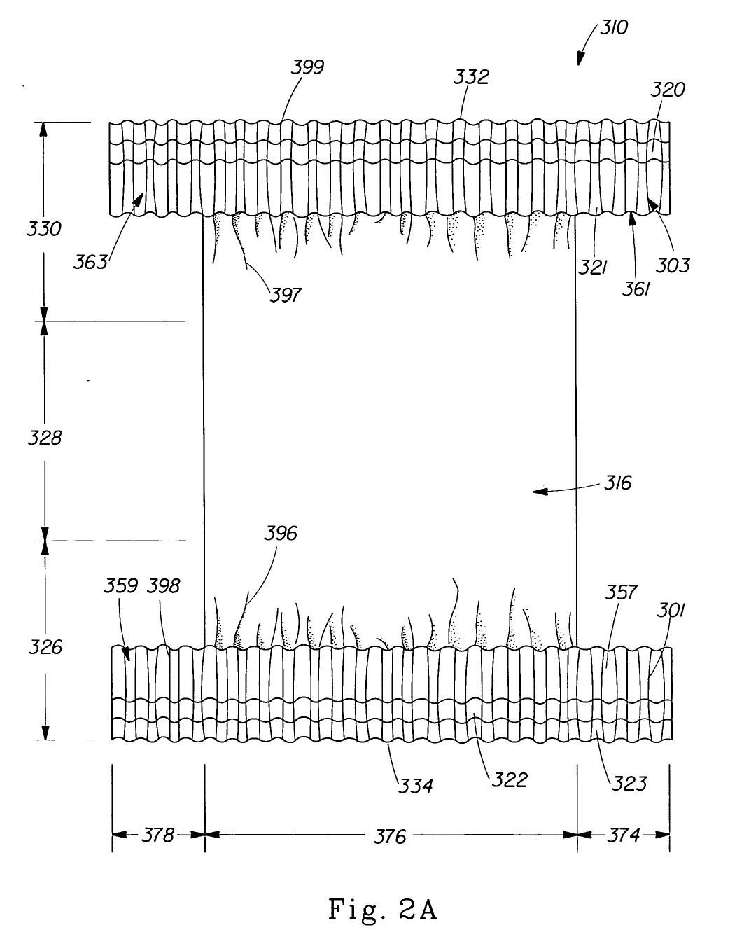

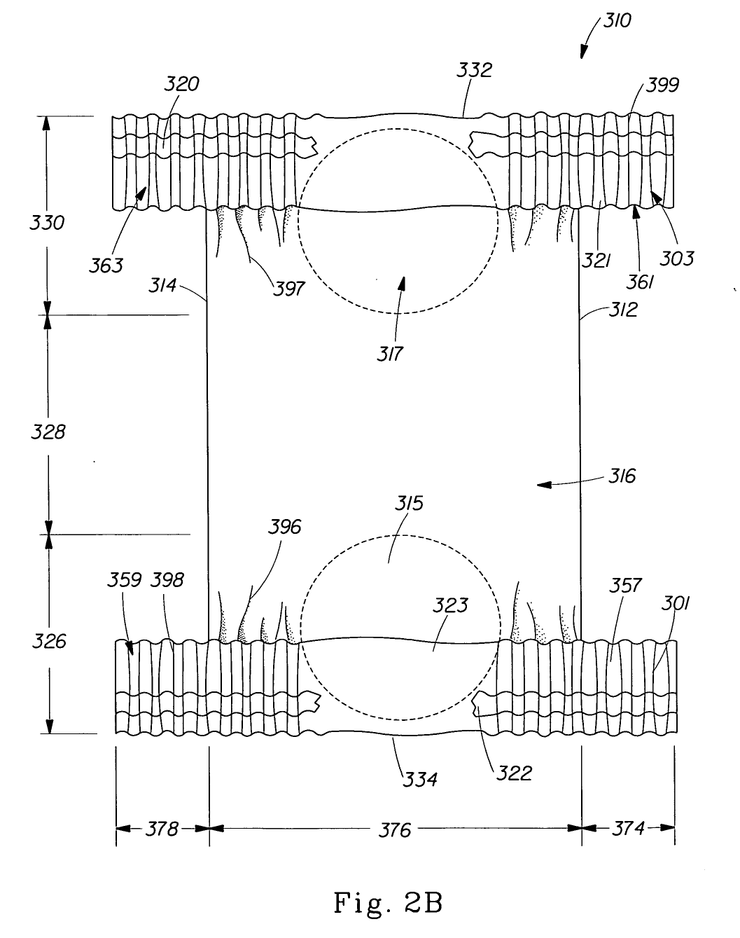

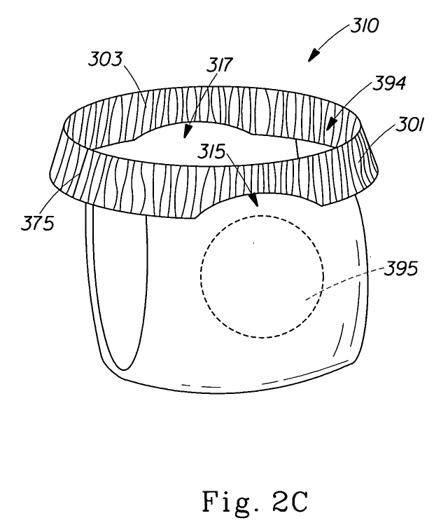

As shown in FIG. 2A, the absorbent article 310 comprises a chassis 316. The chassis 316 comprises front and rear waist regions 326, 330, and a front waist edge 334, and a rear waist edge 332. A crotch region 328 is disposed between the front waist region 326 and the rear waist region 330. The front waist region 326, the rear waist region 330, and the crotch region 328 may each comprise a middle region 376, and a first lateral region 374 and a second lateral region 378. The first lateral region 374 includes a first ear panel 357 and a third ear panel 361. The second lateral region 378 includes a second ear panel 359 and a fourth ear panel 363. The middle region 376 includes an absorbent core (not shown).

An elastomeric first waist member 301 and an elastomeric second waist member 303 can be joined to the chassis 316. The elastomeric first waist member 301 comprises a first elastic member 322 joined to a substrate 323. The first elastic member 322 can be joined to the substrate 323 while under tension such that upon relaxation of the first elastic member 322, the substrate 323 corrugates (see corrugations 398). If the elastomeric first waist member 301 is joined to the absorbent article 310 while the elastomeric first waist member 301 is under tension, then corrugations 396 may form in the front waist region 326 of the absorbent article 310 once tension is released.

Similarly, the elastomeric second waist member 303 comprises a second elastic member 320 joined to a substrate 321. The second elastic member 320 can be joined to the substrate 321 while under tension such that upon relaxation of the second elastic member 320, the second substrate 321 corrugates (see corrugations 399). Also, if the elastomeric second waist member 303 is joined to the absorbent article 310 while under tension, then corrugations 397 may form in the rear waist region 330 of the absorbent article 310.

Regarding FIG. 2B, graphic designs are often included in the front and rear waist regions 326, 330 of the chassis 316. Because corrugations 396, 397 in the front waist region 326 and the rear waist region 330 have a tendency to interfere with graphic designs, the corrugations 396 and 397 are not always desired. In order to minimize the corrugations in the middle region 376 of the front waist region 326, the elastomeric first waist member 301 may further comprise a first waist heat deactivated zone 315 which overlaps and deactivates a portion of the elastomeric first waist member 301. Note that the corrugations 398 on the elastomeric first waist member 301 are minimized in the first waist heat deactivated zone 315. Similarly, the corrugations 396 in the middle region 376 of the front waist region 326 have been minimized. Similar to the elastomeric first waist member 301, the elastomeric second waist member 303 may further comprise a second waist heat deactivated zone 317. The second heat deactivated zone 317 overlaps and deactivates a portion of the elastomeric second waist member 303 in order to minimize corrugations 399 on the elastomeric second waist member 303 as well as the corrugations 397 in the middle region 376 of the rear waist region 330. The elastomeric first and second waist members 301 and 303 may be heat deactivated in the corresponding first and second waist heat deactivated zones 315, 317 before or after the elastomeric first and the second waist members 301, 303 are joined to the chassis 316.

The first waist member 301 may extend beyond a first longitudinal edge 314 to beyond a second longitudinal edge 312 in the front waist region 326. The second waist member 303 may extend from beyond the first longitudinal edge 314 to beyond the second longitudinal edge 312 in the rear waist region 330. The amount by which the first and second waist members 301, 303 extend beyond their respective longitudinal edges varies on many factors. The first and the second waist members 301, 303, should be sized such that the resulting elastic belt member is adaptable to comfortably encircle a waist of a wearer while providing enough tension force about the wearer's waist to hold the absorbent article snugly on the wearer.

As shown in FIG. 2C, the first waist member 301 and the second waist member 303 of the absorbent article 310 can be joined at their respective ends such that an elastomeric belt member 394 is formed which is adaptable for encircling the waist of a wearer. The first waist member 301 and the second waist member 303 can be joined at a first seam 375 and a second seam (not shown). A first graphic design 395 can be placed onto the front waist region because the first waist heat deactivated zone 315 deactivates a portion of the elastomeric first waist member 301 in the middle region of the front waist region. Similarly, a second graphic design (not shown) can be placed onto the rear waist region because the second waist heat deactivated zone 317 deactivates a portion of the elastomeric second waist member in the middle region of the rear waist region.

As discussed above, belt structures may also comprise the heat deactivated zones of the present invention. An example of another belt structure comprises an ear and/or side panel and at least a portion of the waist functionality. In another example, a belt completely encircling a wearer's waist (i.e., a 360 degree belt) may be formed, for example, by depositing one or more laterally oriented elastic members (or an array thereof) adjacent front and rear waist edges so as to form a band of tension about the wearer's waist. Regardless of which embodiment is chosen, the resulting belt structure may comprise a heat deactivated zone.

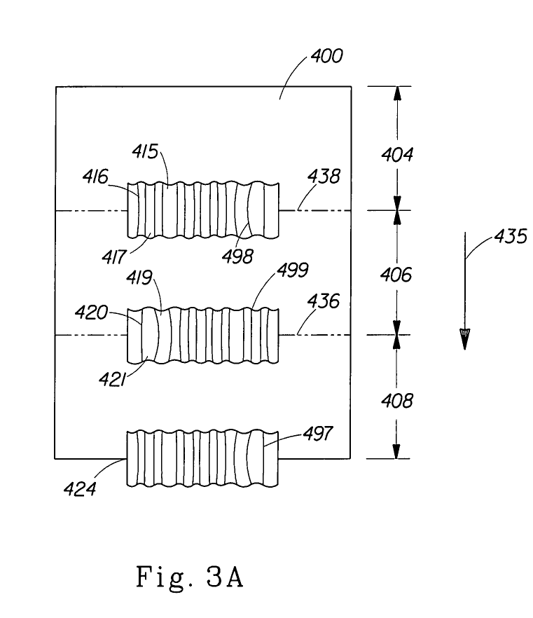

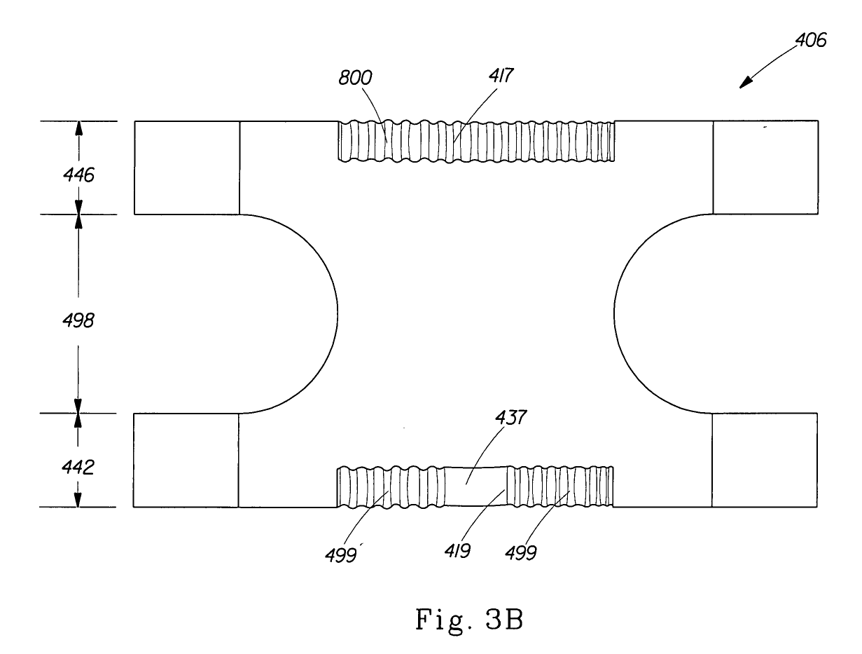

Regarding FIG. 3A, a heat deactivated zone can affect portions of an elastic member or the entire member as discussed previously. As an example, discrete elastomeric laminates 416, 420, and 424, can be joined under tension to a web 400 of absorbent articles such that corrugations 498, 499, and 497 are formed on the discrete elastomeric laminates 416, 420, and 424, respectively. The discrete elastomeric laminates 416, 420, and 424, can comprise an elastic member and a substrate to which that elastic member is joined.

An absorbent article 406 can have a discrete elastomeric laminate 420 on its leading edge 436 and a discrete elastomeric laminate 416 on its trailing edge 438 with respect to a processing direction 435. The discrete elastomeric laminate 416 makes up a first front waist member 415 for an absorbent article 404 and also makes up a first rear waist member 417 for an absorbent article 406. Similarly, the discrete elastomeric laminate 416 makes up a second front waist member 419 for absorbent article 406 and a second rear waist member 421 for an absorbent article 408. The discrete elastomeric laminates comprise an elastic member joined to a substrate.

A cutting process which can separate the absorbent article 404 from the absorbent article 406 can be used to separate the first front waist member 415 and the first rear waist member 417. Similarly, a cutting process which can separate the absorbent article 406 from the absorbent article 408 can be used to separate the second front waist member 419 from the second rear waist member 421.

It may be desirable to have the stretch characteristics of the elastomeric laminate 416 vary, i.e. the second front waist member 419 has different stretch characteristics than the second rear waist member 421. Thus, in another embodiment of the present invention, the discrete elastomeric laminate 416 may comprise a third waist heat deactivated zone which affects the second front waist member 419 or the second rear waist member 421 only.

As shown in FIG. 3B, the absorbent article 406 comprises a front waist region 442, a rear waist region 446, and a crotch region 498 disposed therebetween. The second front waist member 419 may comprise a third waist heat deactivated zone 437 which affects only the second front waist member 419 (see FIG. 3A also) in the front waist region 442. By providing the third waist heat deactivated zone 437 to the second front waist member 419, the stretch characteristics of the second front waist member 419 can be modified such that they are different from the stretch characteristics of the second rear waist member 421 (see FIG. 3A). In addition, the third waist heat deactivated zone 437 minimizes any corrugations 499 formed in the front waist region 442. Note that the third waist heat deactivated zone 437 may only deactivate a portion of the front waist member 419 or may deactivate the entire front waist member 419. Alternative to or in conjunction with the third waist heat deactivated zone 437, a fourth waist heat deactivated zone may deactivate a portion or all of the rear waist member 417 such that corrugations 498 in the rear waist region 446 are minimized.

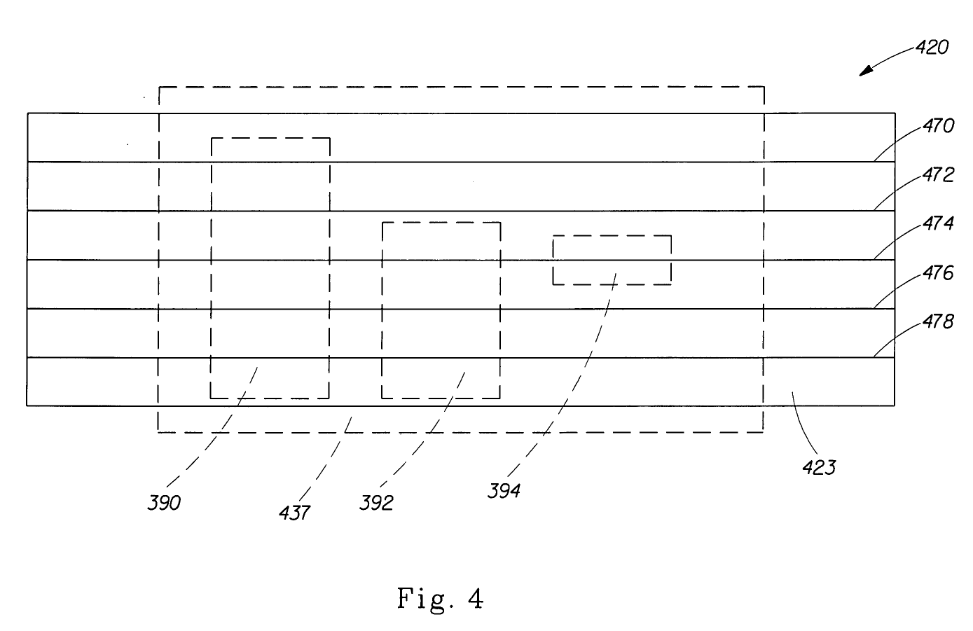

As shown in FIG. 4, the third waist heat deactivated zone 437 may comprise multiple heat deactivation areas, i.e. 390, 392, 394. Similarly, although not shown, all of the heat deactivated zones or waist heat deactivated zones discussed herein may comprise multiple heat deactivated areas as well.

The elastic member of the discrete elastomeric laminate 420 (see also FIG. 3A) may comprise a plurality of elastomeric strands, i.e. 470, 472, 474, 476, and 478 which are joined to a substrate 423. A heat deactivated area can deactivate a plurality of elastomeric strands or a single elastomeric strand. For example, a first heat deactivated area 390 can deactivate all of the elastomeric strands 470, 472, 474, 476, and 478 of the discrete elastomeric laminate 420. As another example, a second heat deactivated area 392 can deactivate a smaller number of elastomeric strands, specifically, 474, 476, and 478. In yet another example, a third heat deactivated area 394 can deactivate a single strand, i.e. 474.

The previously discussed embodiments are provided as specific examples of where heat deactivation zones can relax, i.e. deactivate, elastic members in an absorbent article. However, the heat deactivation zones can deactivate elastic members in many different regions of the absorbent article and are not limited to the areas or regions previously discussed. Examples provided below demonstrate many of the areas in which a heat deactivated zone can be utilized.

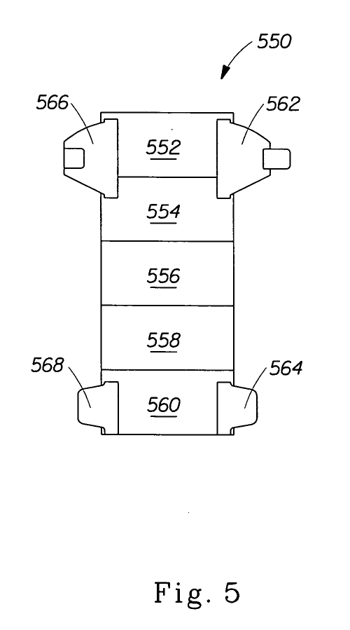

As shown in FIG. 5, an absorbent article 550 comprising multiple regions, i.e. 552, 554, 556, 558, 560, 562, 564, 566, and 568, may each comprise a heat deactivated zone. The absorbent article 550 may include a liquid impermeable backsheet and an outer cover made of a nonwoven material. The backsheet may comprise the heat deactivated zones described above such that the stretch properties of regions 552, 554, 556, 558, and 560 are different from one another. Specifically, the heat deactivated zones may be utilized in these regions in order to best accommodate the curvature of the wearer's body. In certain cases for purposes of enhancing fit on a wearer, the various stretch properties are symmetrical in that regions 552 and 560 have similar stretch properties, regions 554 and 558 also have similar stretch properties while region 556 has a third type of stretch property. It should be understood, however, that this is not necessary and that individual regions 552, 554, 556, 558 and 560 may vary individually and widely in terms of properties, size, and shape, without deviating from the scope of the invention.

Similarly, the regions 562, 564, 566, and 568 can utilize heat deactivated zones to accommodate the curvature of the wearer. These areas, typically referred to as “ear panels”, act to secure the article about the waist of the wearer. Thus, heat deactivated zones can be utilized to modify the stretch properties of one or more of the ear panels or a portion thereof in order to provide a better fitting article.

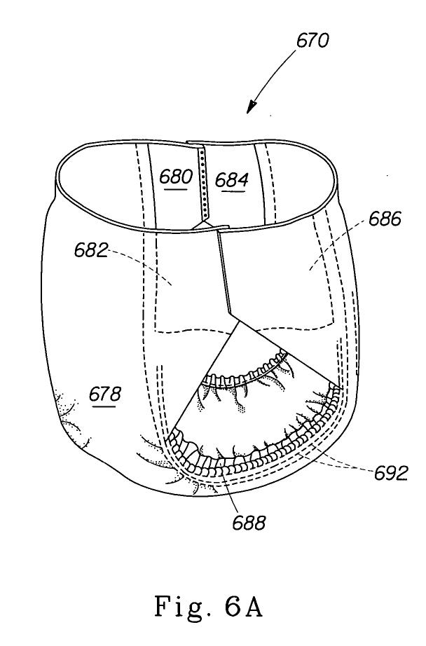

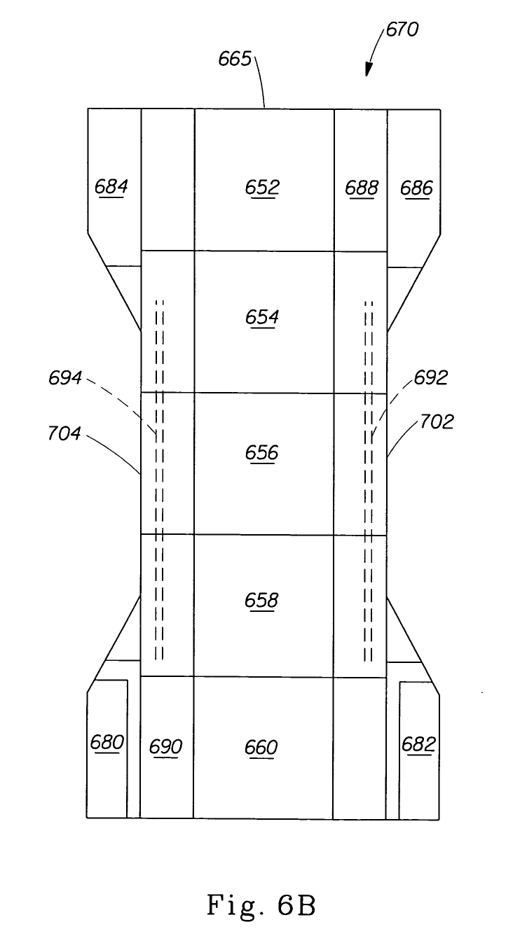

FIG. 6A shows a pant 670 which may comprise one or more heat deactivated zones. The heat deactivated zones of the present invention may be utilized anywhere on the pant diaper 670 where an elastic laminate (an elastic joined to a substrate) exists. An outer cover 678 comprising a nonwoven can be disposed on the outer surface of the pant diaper 670. The outer cover 678 can be joined to a backsheet so as to form a portion of a pair of leg openings and a waist opening for the wearer. Leg elastics 692 and 694 (see FIG. 6B) can provide a more comfortable fit for a wearer which can extend beyond a single region of the absorbent article 670. Also, two pair of side panels 680, 682, and 684, 686 are attached to the outer cover 678. The pant 670 may further comprise barrier leg cuffs 688, 690 which can extend from end edge 665 to end edge 669 (see FIG. 6B).

As shown in FIG. 6B, two of the side panels 680 and 684 extend outward from a longitudinal edge 704. The other two side panels 686 and 682 extend outward from a longitudinal edge 702. The heat deactivated zones of the present invention may be incorporated into the two pair of side panels 680, 682, and 684, 686 in order to accommodate the contours of a wearer's body or to modify the stretch properties of a particular side panel. The heat deactivated zones of the present invention may also be incorporated into pant 670 in a waist feature (not shown) that is disposed adjacent end edges 665 and 669 or barrier leg cuffs 688 and 690.

A heat deactivated zone described in the present invention may be utilized in any region of the absorbent article 670, i.e. 652, 654, 656, 658, and 660. The backsheet may utilize a heat deactivated zone such that the stretch properties of regions 652, 654, 656, 658, and 660 are different from one another. Specifically, the heat deactivated zones may be utilized in these regions in order to best accommodate the curvature of the wearer's body.

In order to heat deactivate an elastic member or a waist member as described herein, the elastic member or waist member should be under tension and extended while thermal energy is applied. The exact temperatures and amount of time required for the deactivation to take place depend greatly upon the chemistry of the elastic members, the type of thermal energy source, the type of substrate the elastic members are bonded to, the porosity of the substrate to which the elastic members are bonded to, and the mode by which thermal energy is added to the elastic member. Provided below is an example of a time and a temperature which will produce a deactivated zone in an elastomeric laminate.

As discussed previously, when heat is applied to an elastic member, the tensile strength of the elastic member can decrease and become less than the contraction force thereby causing the elastic member to break. In the present invention, heat can be applied by any thermal energy source known in the art which does not contravene the disclosed invention. In addition, a thermal energy source can be used to heat an elastic member in any region of the absorbent article. The thermal energy can be applied in several modes or combinations of these modes, i.e. conduction, convection, or radiation.

A sample for demonstrating the heat deactivation process may be constructed as follows. A sample comprising an elastomeric laminate having elastic strands between a first and a second nonwoven layer can be created. The elastic strands can have a diameter of about 0.23 mm and be spaced apart at about 1.5 mm. For testing purposes, the sample should be large enough such that a difference between a heat deactivated zone and the unaffected areas of the laminate can be seen. The elastic strands can be made from a Kraton® G1730 formulation. Each nonwoven layer can be a polyester carded nonwoven with a basis weight of 20 gsm each. The laminate can be extended and held at about 200% of its original length (twice its original length) while thermal energy is applied to the elastomeric laminate. The elastic strands can be deactivated in about 1 second using a general convective thermal energy source which provides air at about 110° C. at a pressure of about 0.5 bar.

Note that the length to which the elastomeric laminate is extended can affect the time for deactivation. As previously discussed, when heat is applied to an extended elastomeric laminate, the contractive force can become higher than the tensile force of an elastic member within the laminate. Thus, the elastic member in an elastomeric laminate extended to approximately 150% its original length (1.5 times its original length) may be deactivated slower than the elastic member in an elastomeric laminate which is extended to approximately 250% its original length (2.5 times its original length).

Any elastic members disclosed herein may be of various shapes and sizes. The elastic members may comprise a plurality of elastomeric strands. Furthermore, the elastomeric strands may be made from liquid elastic that is extruded through a die to achieve the desired strand elastic diameter and/or shape. The shape of the extruded elastomeric strands is not limited. For example, typical elastomeric strands have a circular cross sectional shape, but sometimes the plurality of elastomeric strands may have different shapes, such as a trilobal shape, or a flat (i.e., “ribbon” like) shape. Suitable elastomeric strand shapes (not all shown) include rectangles, circles, ellipses, diamonds, triangles, parallelograms, trapezoids, wedges or other sections of circles or ellipses, other polygons, or other irregular enclosed shapes. Furthermore, the thickness or diameter of the elastomeric strands may vary in order to accommodate a particular application. Typically, the thickness of elastomeric strands may be in the range of about 0.02 mm to about 1 mm.

Furthermore, any elastic members described herein in a heat deactivated zone for the absorbent articles of the present invention mentioned heretofore, should have a lower glass transition temperature than the melting temperature of the substrate to which it is joined. For instance, both the first elastic member and the second elastic member, as described herein, should have a lower glass transition temperature than the melting temperature of a topsheet, a backsheet, or a substrate to which they are joined. An example of a suitable material for an elastic member is a resilient thermoplastic material that is a styrene block copolymer. Examples of suitable styrene block copolymers are (1) a polymer resin sold under the trade name Vector® manufactured by the Dexco Corporation© located in Houston, Tex.; (2) a polymer resin sold under the trade name Septon® manufactured by Kuraray Co.® Ltd. located in Pasadena, Tex.; (3) a polymer resin sold under the trade name Kraton D® or Kraton G® manufactured by Kraton Polymers® located in Houston, Tex.

In general, the elastic members disclosed herein are joined to a substrate, a topsheet, or a backsheet, while under tension thereby allowing the resulting laminate structure to be elastically extensible. Generally, this process is termed “live stretch.” Alternatively, the elastic members may be joined to a substrate while in a relaxed state. However, the resulting laminate structure could be activated to be made elastically extensible. Generally, this process is termed “activation” or “mechanical activation”, as described previously. Both the live stretch process and the mechanical activation process are further described in U.S. application Ser. No. 10/397,334, filed on Mar. 26, 2003 and entitled ELASTOMERIC NONWOVEN LAMINATES AND PROCESS FOR PRODUCING SAME.

An example of a suitable material for a substrate is a nonwoven material comprising fibers of polypropylene, polyethylene, polyester, polystyrene, or any combination thereof. Some suitable nonwoven materials and suitable topsheets are a 10 gsm SMS nonwoven from Avgol Nonwovens Industries, located in Holon, Israel, a 13 gsm spunbond nonwoven from Fibretec, and a 27 gsm carded nonwoven from BBA Fiberweb™ located in Nashville, Tenn. A suitable backsheet is a 27 gsm carded nonwoven from BBA Fiberweb™. Further examples of suitable materials for topsheets and backsheet are discussed further in U.S. Pat. No. 6,004,306 to the extent that these examples do not contravene the requirements of the disclosed invention.

The adhesive disclosed herein may be applied to the absorbent article in a spiral pattern, or the adhesive may comprise discrete bond sites. Suitable adhesives for joining an elastic member to a substrate or the chassis as described herein are manufactured by Bostik Findley®, located in Scranton, Pa., and sold under the trade names H-2031 and H-20401. Alternatively, the elastic members disclosed herein can be joined to the absorbent article or their respective substrate by any means known in the art which does not contravene the disclosed invention.

The heat deactivated zones of the present invention may be utilized in diapers or diaper components which may take any one or more of the materials, designs, and methods of assembly described hereinafter without departing from the scope of the present invention. While any of the article components may be assembled in a variety of well known configurations, exemplary diaper configurations are described generally in U.S. Pat. No. 3,860,003; U.S. Pat. No. 5,151,092; and U.S. Pat. No. 5,221,274; and U.S. Pat. No. 5,554,145; U.S. Pat. No. 5,569,234; U.S. Pat. No. 5,580,411; and U.S. Pat. No. No. 6,004,306.

The absorbent articles described herein can utilize a variety of materials in their manufacture. Some examples of the materials which can be used in the manufacture of absorbent articles are provided below; however, the list of materials provided is by no means exhaustive. For example, breathable materials, which are used extensively in absorbent articles may include materials such as woven webs, nonwoven webs, composite materials such as film-coated nonwoven webs, microporous films such as manufactured by Mitsui Toatsu Co., of Japan under the designation ESPOIR NO™ and by Exxon Chemical Co., of Bay City, Tex., under the designation EXXAIRE™, and monolithic films such as manufactured by Clopay Corporation, Cincinnati, Ohio under the name HYTREL™ blend P18-3097. Some breathable composite materials are described in greater detail in U.S. Pat. No. 6,187,696; U.S. Pat. No. 5,938,648; U.S. Pat. No. 5,865,823; and U.S. Pat. No. 5,571,096.

Exemplary absorbent structures for use as the absorbent core are described in U.S. Pat. No. 4,610,678; U.S. Pat. No. 4,673,402; U.S. Pat. No. 4,834,735; U.S. Pat. No. 4,888,231; U.S. Pat. No. 5,137,537; U.S. Pat. No. 5,147,345; U.S. Pat. No. 5,342,338; U.S. Pat. No. 5,260,345; U.S. Pat. No. 5,387,207; and U.S. Pat. No. 5,625,222.

Suitable absorbent and nonabsorbent sublayers are described in European Patent Application No. EP 0 847 738 A1 and U.S. Pat. No. 5,941,864. Further, the sublayer, or any portion thereof, may include or be coated with a lotion or other known substances to add, enhance or change the performance or other characteristics of the element.

Some exemplary surface fastening systems are disclosed in U.S. Pat. No. 3,848,594; U.S. Pat. No. B1 4,662,875; U.S. Pat. No. 4,846,815; U.S. Pat. No. 4,894,060; U.S. Pat. No. 4,946,527; the herein before referenced U.S. Pat. No. 5,151,092; and U.S. Pat. No. 5,221,274. An exemplary interlocking fastening system is disclosed in co-pending U.S. application Ser. No. 09/143,184 entitled “Absorbent Article Fastening Device” in the names of Kline et al. filed on Aug. 8, 1998. The fastening system may also: provide a means for holding the article in a disposal configuration as disclosed in U.S. Pat. No. 4,963,140; include primary and secondary fastening systems, as disclosed in U.S. Pat. No. 4,699,622; means to reduce shifting of overlapped portions or to improve fit as disclosed in U.S. Pat. No. 5,242,436. means to resist gapping at a wearer's belly as disclosed in U.S. Pat. No. 5,499,978 in U.S. Pat. No. 5,507,736 and in U.S. Pat. No. 5,591,152.

Examples of diapers with elasticized side panels are disclosed in U.S. Pat. No. 4,857,067; U.S. Pat. No. 4,381,781; U.S. Pat. No. 4,938,753; the herein before referenced U.S. Pat. No. 5,151,092; U.S. Pat. No. 5,221,274; U.S. Pat. No. 5,669,897; and U.S. Pat. No. 6,004,306.

Embodiments of the present invention may also include pockets for receiving and containing waste, spacers which provide voids for waste, barriers for limiting the movement of waste in the article, compartments or voids which accept and contain waste materials or any combinations thereof. Examples of pockets and spacers for use in absorbent products are described in U.S. Pat. No. 5,514,121; U.S. Pat. No. 5,171,236; U.S. Pat. No. 5,397,318; U.S. Pat. No. 5,540,671; U.S. Pat. No. 6,168,584; U.S. Pat. No. 5,306,266; and U.S. Pat. No. 5,997,520. Examples of compartments or voids are disclosed in U.S. Pat. No. 4,968,312; U.S. Pat. No. 4,990,147; U.S. Pat. No. 5,062,840; and U.S. Pat. No. 5,269,755. Examples of suitable transverse barriers are described in U.S. Pat. No. 5,554,142; U.S. Pat. No. 6,010,490; and U.S. Pat. No. 5,653,703. Examples of other structures especially suitable for management of low viscosity feces are disclosed in U.S. Pat. Nos. 5,941,864, 5,977,430, and 6,013,063.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article comprising:
a chassis having a first longitudinal edge, a second longitudinal edge, a front waist edge, a rear waist edge, a front waist region, a rear waist region, and a crotch region disposed therebetween, the chassis further comprising:
a topsheet;
a backsheet joined to the topsheet; and
an absorbent core disposed between the topsheet and the backsheet;
a first elastic member joined under tension to the chassis proximate the first longitudinal edge; and
a first heat deactivated zone overlapping the first elastic member in at least a portion of the front waist region or the rear waist region minimizing corrugations in the front or rear waist region.

2. The absorbent article of claim 1 further comprising a second elastic member joined under tension to the chassis proximate the second longitudinal edge such that corrugations are formed in the chassis in the front, rear, and crotch regions inboard of the second elastic member upon relaxation of the second elastic member, and a second heat deactivated zone overlapping the second elastic member in at least a portion of the front waist region or the rear waist region.

3. The disposable absorbent article of claim 2, wherein the first and the second heat deactivated zones overlap the first and second elastic members in the portion of the front waist region.

4. The disposable absorbent article of claim 3 further comprising a third heat deactivated zone overlapping the first elastic member in at least a portion of the rear waist region, and a fourth heat deactivated zone overlapping the second elastic member in at least a portion of the rear waist region.

5. The disposable absorbent article of claim 3 further comprising:
a first ear panel extending outboard of the absorbent core toward the first longitudinal edge, and
a second ear panel extending outboard of the absorbent core toward the second longitudinal edge.

6. The disposable absorbent article of claim 5, wherein the first ear panel and the second ear panel each comprise a portion of the topsheet and a portion of the backsheet, wherein both portions are proximate the front waist edge.

7. The disposable absorbent article of claim 6, wherein the first elastic member and the second elastic member are each adhesively bonded to the chassis between the topsheet and the backsheet throughout the front waist region proximate the front waist edge such the first and the second ear panels are reinforced.

8. The disposable absorbent article of claim 1, wherein the first elastic member and the second elastic member both comprise a plurality of elastomeric strands.

9. The disposable absorbent article of claim 5, wherein the first ear panel comprises a portion of a proximal end of a first barrier leg cuff and a portion of the backsheet, and the second ear panel comprises a portion of a proximal end of a second barrier leg cuff and a portion of the backsheet, wherein all portions are proximate the front waist edge.

10. The disposable absorbent article of claim 9, wherein the first elastic member is adhesively joined to the chassis among the topsheet, backsheet, and the first barrier leg cuff, and the second elastic member is adhesively joined to the chassis among the topsheet, the backsheet, and the second barrier leg cuff, each being joined throughout the front waist region proximate the front waist edge such that the first and the second ear panels are reinforced.

11. The disposable absorbent article of claim 1, wherein the chassis further comprises a first lateral region, a second lateral region, and a middle region between the first and second lateral regions, wherein the disposable absorbent article further comprises:
an elastomeric first waist member joined under tension to the front waist region of the chassis, wherein the elastomeric first waist member comprises a first waist heat deactivated zone disposed in a portion of the elastomeric first waist member minimizing corrugations in the portion of the elastomeric first waist member, the portion being joined to the middle region of the chassis.

12. A disposable absorbent article comprising:
a chassis having a front waist edge, a rear waist edge, and a front waist region and a rear waist region adjacent to their respective front and rear waist edges, and a crotch region between the front and rear waist regions, wherein the first waist region, the rear waist region, and the crotch region each include a first lateral region, a second lateral region, and a middle region between the first and second lateral regions, and wherein the chassis further comprises a topsheet joined to a backsheet, and an absorbent core disposed between the topsheet and the backsheet; and
an elastomeric first waist member joined under tension to the front waist region of the chassis, wherein the elastomeric first waist member comprises a first waist heat deactivated zone overlapping the elastomeric first waist member in the middle region of the front waist region minimizing corrugations in the middle region of the front waist region.

13. The disposable absorbent article of claim 12 further comprising an elastomeric second waist member joined under tension to the rear waist region of the chassis, wherein the elastomeric second waist member comprises a second waist heat deactivated zone overlapping the elastomeric second waist member in the middle region of the rear waist region minimizing corrugations in the middle region of the rear waist region are minimized.

14. The disposable absorbent article of claim 13, wherein the elastomeric first and second waist members transcend a first longitudinal edge and a second longitudinal edge.

15. The disposable absorbent article of claim 14, wherein the elastomeric first waist member is joined to the elastomeric second waist member thereby forming an elastic belt member adaptable to encircle a waist of a wearer.

16. The disposable absorbent article of claim 12, wherein the elastomeric first and second waist members are discrete members and do not transcend a first longitudinal edge and a second longitudinal edge.

17. A method for making a disposable absorbent article, the method comprising the steps of:
providing a chassis having a first longitudinal edge, a second longitudinal edge, a front waist edge, a rear waist edge, a front waist region, a rear waist region, a crotch region disposed therebetween, and an absorbent core disposed between a topsheet and a backsheet;
joining a first elastic member under tension to the chassis proximate the first longitudinal edge such that corrugations are formed in the chassis in the front, rear, and crotch regions inboard of the first elastic member upon relaxation of the first elastic member;
joining a second elastic member under tension to the chassis proximate the second longitudinal edge such that corrugations are formed in the chassis in the front, rear, and crotch regions inboard of the second elastic member upon relaxation of the second elastic member;
heat deactivating a portion of the first elastic member while under tension proximate to the front waist edge; and
heat deactivating a portion of the second elastic member while under tension proximate to the front waist edge.

17. The method of claim 16 further comprising the steps of heat deactivating a portion of the first elastic member while under tension proximate to the rear waist edge, and heat deactivating the second elastic member while under tension proximate to the rear waist edge.

18. A method for making a disposable absorbent article having a chassis having a front waist edge, a rear waist edge, and a front waist region and a rear waist region adjacent to their respective front and rear waist edges, and a crotch region between the front and rear waist regions, wherein the first waist region, the rear waist region, and the crotch region each include a first lateral region, a second lateral region, and a middle region between the first and second lateral regions, and wherein the chassis further comprises a topsheet joined to a backsheet, and an absorbent core disposed between the topsheet and the backsheet, the method comprising the steps of:
providing a first waist member comprising a first elastic member joined under tension to a substrate such that upon relaxation, the first waist member corrugates;
heat deactivating at least a portion of the first waist member over the first elastic member while under tension such that corrugations in the portion of the first waist member are minimized when the first waist member is relaxed; and
joining the first waist member under tension to the front waist region such that in a relaxed state corrugations in the middle region of the front waist region are minimized.

19. The method of claim 18 further comprising the steps of:
providing a second waist member comprising a second elastic member joined under tension to a substrate such that upon relaxation, the second waist member corrugates;
heat deactivating at least a portion of the second waist member over the second elastic member while under tension such that corrugations in the portion of the second waist member are minimized when the second waist member is relaxed, and
joining the second waist member under tension to the rear waist region such that in a relaxed state, corrugations in the middle region of the rear waist region are minimized.

20. The method of claim 19, wherein the first waist member can be joined to the second waist member thereby forming an elastic belt member adaptable to encircle a waist region of a wearer.
